(19) United States
(12) Patent Application Publication
(10) Pub. No.: US 2018/0333066 A1
(43) Pub. Date: Nov. 22, 2018

(54) APPARATUS FOR MEASURING ELECTROENCEPHALOGRAM, SYSTEM AND METHOD FOR DIAGNOSING AND PREVENTING DEMENTIA

(71) Applicant: GI Signal, Ltd., Busan (KR)

(72) Inventors: Hee Kyong YOO, Seoul (KR); Won Bae KIM, Gwacheon-si Gyeonggi-do (KR)

(73) Assignee: GI Signal, Ltd., Busan (KR)

(21) Appl. No.: 15/633,367

(22) Filed: Jun. 26, 2017

(30) Foreign Application Priority Data
May 17, 2017 (KR) 10-2017-0061005

(51) Int. Cl. A61B 5/0478; A61B 5/0484; A61B 5/00; A61B 5/04

(52) U.S. Cl. CPC A61B 5/0478; A61B 5/04012; A61B 5/4088; A61B 5/04842

(57) ABSTRACT

An apparatus for measuring electroencephalogram (EEG) may include at least one an EEG-detecting electrode and an eye patch. The EEG-detecting electrode may be configured to detect an EEG of a subject. The eye patch may be configured to shut eyes of the subject when measuring the EEG.

### CROSS-REFERENCES TO RELATED APPLICATION

The present application claims priority under 35 U.S.C. § 119(a) to Korean application number 10-2017-0061005, filed on May 17, 2017, in the Korean Intellectual Property Office, which is incorporated herein by reference in its entirety.

### BACKGROUND

### 1. Technical Field

Various embodiments generally relate to an apparatus for measuring electroencephalogram, a system and a method for diagnosing and preventing dementia, more particularly to an apparatus for measuring accurate electroencephalogram signals, a system and a method for diagnosing and preventing dementia.

### 2. Related Art

Technologies for diagnosing health of a human body using bio-signals may have been widely studied.

The bio-signals may include electroencephalogram (EEG), electromyogram (EMG), electrocardiography (ECG), etc. When a stimulus may be applied to a cerebral cortex, an ionized current may flow through a neuron to form an electric field and a magnetic field. A microcurrent change may be measured using an electrode on a scalp to form a waveform. The waveform may be correspond to the EEG. The EEG may have a about 0 Hz to 100 Hz of frequency band. Because the current change may be dozens of μN, the current change may be amplified. The amplified current change may be recorded as the EEG.

The EEG may be classified into a delta(δ) wave of no more than about 4 Hz, a theta(θ) wave of about 4 Hz to 8 Hz, an alpha(α) wave of about 8 Hz to 12 Hz, a beta(3) wave of about 12 Hz to 30 Hz, and a γ wave of about 30 Hz to about 50 Hz in accordance with activation state of a brain, i.e., a vibrated frequency range.

The EEG may be used for diagnosing sleep, awake condition and brain abnormalities. Recently, diagnosis of dementia using the EEG may be widely developed.

A digital EEG measuring instrument such as a brainwave sensor may be developed. The dementia may be diagnosed by analyzing and applying the electroencephalography. However, in order to accurate make a diagnosis, a long skilled observer or a clinical expert may be required. Further, the skilled observers may have different judgment standards.

Further, the EEG of a subject with opening of the eyes may be measured. Although the eyes of the subject may be closed, the eyes of the subject may be blinked by stimulus. This eye blinking may cause generations of artifact as well as the EEG of the subject so that accurate EEG may not be obtained.

### SUMMARY

In an embodiment, an apparatus for measuring electroencephalogram (EEG) may include at least one an EEG-detecting electrode and an eye patch. The EEG-detecting electrode may be configured to detect an EEG of a subject. The eye patch may be configured to shut eyes of the subject when measuring the EEG.

In an embodiment, a system for diagnosing and preventing dementia may include an EEG-measuring apparatus and a dementia-diagnosing apparatus. The EEG-measuring apparatus may include at least one an EEG-detecting electrode, an eye patch and an EEG-stimulating unit. The EEG-detecting electrode may be attached to a head of a subject to detect an EEG of the subject. The eye patch may be configured to shut eyes of the subject when measuring the EEG. The EEG-stimulating unit may be arranged adjacent to the eye patch to provide the subject with a visual stimulus. The dementia-diagnosing apparatus may receive an EEG signal from the EEG-measuring apparatus to determine whether the EEG signal may be within a reference index range or not. The dementia-diagnosing apparatus may output a control command to the EEG-measuring apparatus to provide an abnormal subject having the EEG signal beyond the reference index range with a visual stimulus corresponding to an EEG of a normal subject having the EEG signal within the reference index range.

In an embodiment, according to a method of diagnosing and preventing dementia, a reference index may be set based on a stable EEG of a normal person and an EEG signal in visually stimulating the normal person. An index of a subject may be obtained from a stable EEG of the subject and an EEG signal in visually stimulating the subject. The index of the subject may be compared with the reference index to determine whether the subject may have the dementia or not. The EEG of the subject having the dementia may be stimulated using a signal based on the EEG in visually stimulating the normal person to synchronize the EEG of the subject having the dementia with the EEG of the normal person, thereby preventing the dementia.

### BRIEF DESCRIPTION OF THE DRAWINGS

FIG. 1 is a block diagram illustrating a system for diagnosing and preventing dementia in accordance with example embodiments;

FIG. 2 is a front perspective view illustrating an apparatus for measuring an EEG in accordance with example embodiments;

FIG. 3 is a rear perspective view illustrating an apparatus for measuring an EEG in accordance with example embodiments;

FIG. 4 is an image illustrating a brain of a human being;

FIG. 5 is a block diagram illustrating an apparatus for measuring an EEG in accordance with example embodiments;

FIG. 6 is a perspective view illustrating an apparatus for measuring an EEG worn on a head of an human being in accordance with example embodiments;

FIG. 7 is a block diagram illustrating an apparatus for diagnosing dementia in accordance with example embodiments;

FIG. 8 is a flow chart illustrating operations for an EEG-measuring apparatus in accordance with example embodiments;

FIG. 9 is a flow chart illustrating operations for a dementia-diagnosing apparatus in accordance with example embodiments;

FIG. 10 is a flow chart illustrating operations for a dementia-diagnosing apparatus in accordance with example embodiments; and

FIG. 11 is a flow chart illustrating operations for a dementia-diagnosing and preventing system in accordance with example embodiments.

### DETAILED DESCRIPTION

Hereinafter, example embodiments will be described below with reference to the accompanying drawings through various examples of embodiments.

System for Diagnosing and Preventing Dementia

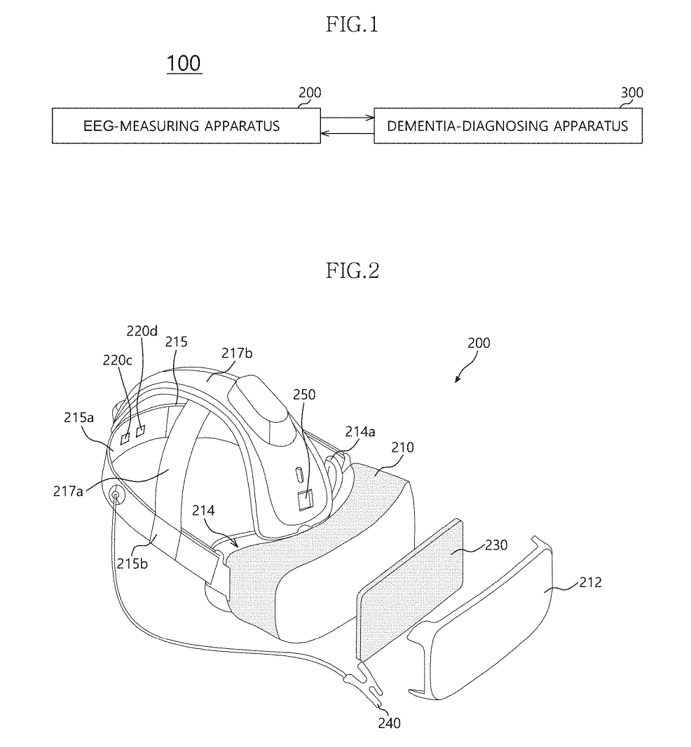

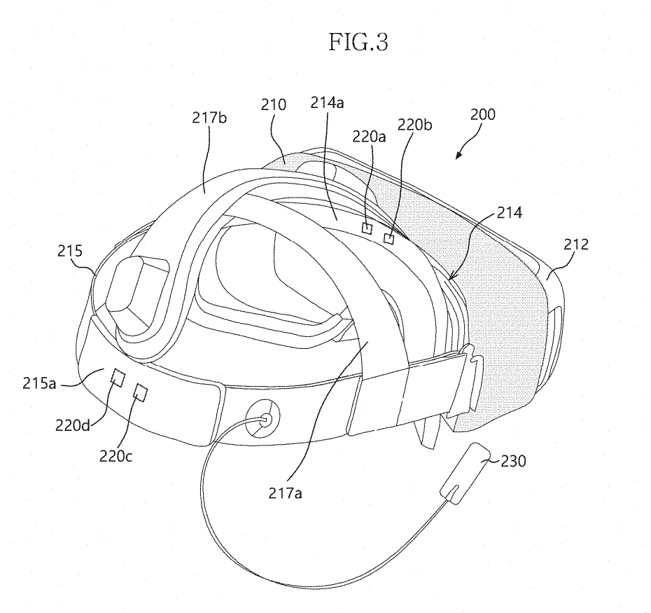

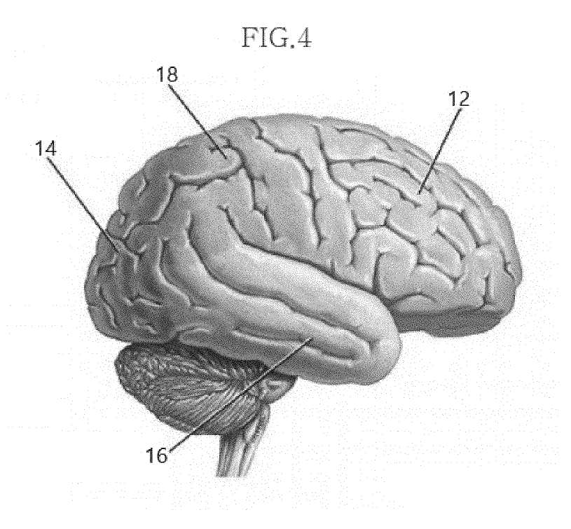

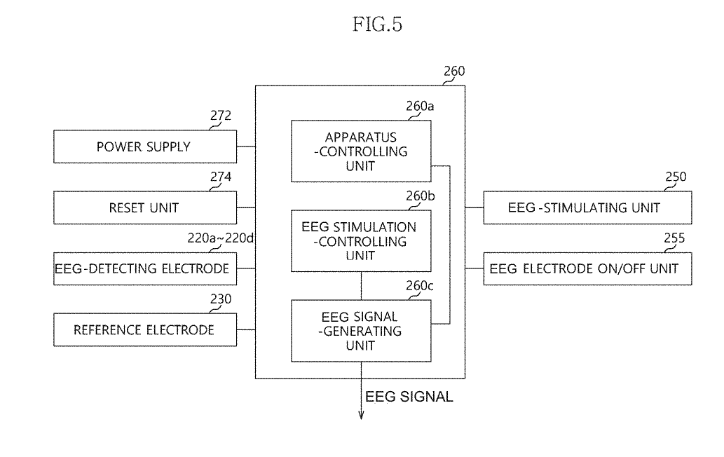

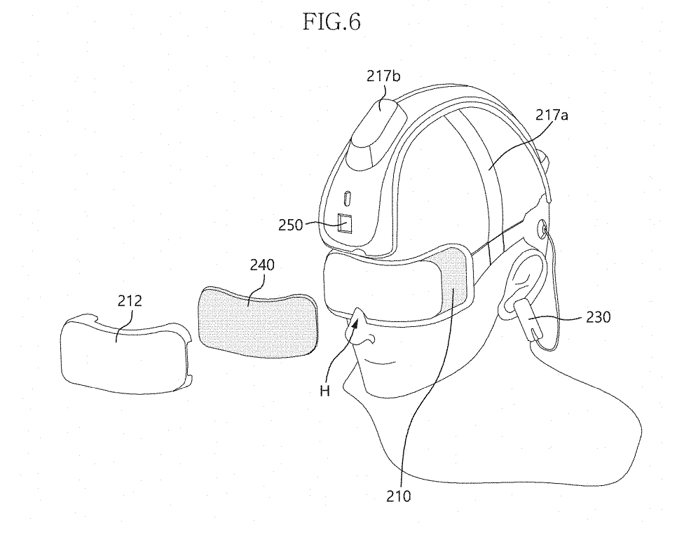

FIG. 1 is a block diagram illustrating a system for diagnosing and preventing dementia in accordance with example embodiments, FIG. 2 is a front perspective view illustrating an apparatus for measuring an EEG in accordance with example embodiments, FIG. 3 is a rear perspective view illustrating an apparatus for measuring an EEG in accordance with example embodiments, FIG. 4 is an image illustrating a brain of a human being, FIG. 5 is a block diagram illustrating an apparatus for measuring an EEG in accordance with example embodiments, and FIG. 6 is a perspective view illustrating an apparatus for measuring an EEG worn on a head of an human being in accordance with example embodiments.

Referring to FIGS. 1 to 6, a system 100 for diagnosing and preventing dementia in accordance with example embodiments may include an electroencephalogram (EEG) measuring apparatus 200 and a dementia-diagnosing apparatus 300.

The EEG-measuring apparatus 200 may include a main frame 210, a wearing unit 215, at least one an EEG-detecting electrode 220a-220d, a reference electrode 230, an eye patch 240, an EEG-stimulating unit 250, an EEG stimulation on/off unit 255 and a controller 260.

The main frame 210 may be worn on a portion of a face and a portion of a head in a subject. The main frame 210 may be supported by the face of the subject using various fixing structures. The main frame 210 may have a light material for providing the subject with comfortable wearing feeling. Further, the main frame 210 may include a material for supporting the eye patch 240.

The main frame 210 may include a window configured to receive the eye patch 240. The main frame 210 may include a front case 212 corresponding to an appearance of the eye patch 240. The front case 212 may function as to protect the eye patch 240 and to prevent a separation of the eye patch 240 from the main frame 210.

The main frame 210 may have a rear surface 214 with which the face of the subject may make contact. The rear surface 214 may be referred to as a face contact portion. The face contact portion 214 may have a shape corresponding to curvatures of the face of the subject. A portion of the face contact portion 214 may include a resilient member 214a. The face contact portion 214 may be configured to surround the window. When the face contact portion 214 may make contact with the face of the subject, the resilient member 214a may include at least one cushion material such as a sponge for providing the subject with comfortable wearing feeling. The resilient member 214a may be detachably attached to the main frame 210 using an adhesive member. Thus, when a plurality of subjects may use the EEG-measuring apparatus 200, the resilient member 214a corresponding to a shape of a face of any one among the subjects may be used. Further, when the resilient member 214a may be contaminated or damaged, the resilient member 214a may be substituted for new one.

The EEG-measuring apparatus 200 worn on the face and the head of the subject may be configured to cover the eyes of the subject. The EEG-measuring apparatus 200 may have a nose recess H (See FIG. 6) configured to receive a nose of the subject.

The wearing unit 215 may be partially combined with the main frame 210. The main frame 210 may be fixed to the face of the subject by the wearing unit 215. The wearing unit 215 may include a band. A portion of the band may include a resilient material. The main frame 210 may be closely positioned to the eyes of the subject by adjusting a length of the band in the wearing unit 215.

The wearing unit 215 may include a first support 215a and a second support 215b. The first support 215a may be arranged at a position corresponding to an occipital lobe of the subject. The second support 215b may be arranged at a position corresponding to a temporal lobe of the subject. However, the positions of the first support 215a and the second support 215b may not be restricted within the above-mentioned positions. Thus, the wearing unit 215 may be configured to surround a portion of the occipital lobe of the subject. The first support 215a and the second support 215b may include a cushion for providing the subject with the comfortable wearing feeling. The first support 215a may be configured to partially receive the EEG-detecting electrodes 220a-220d. The first support 215a may function as to support the EEG-detecting electrodes 220a-220d on the head of the subject. Any one of the first support 215a and the second support 215b may have a function for adjusting the length of the band to provide the wearing unit 215 with a circumferential length of the head of the subject. The any one of the first and second supports 215a and 216b may include a velcro, a magnet, etc. Alternatively, the wearing unit 215 may include eyeglass temples, helmets, straps, etc.

When the EEG-measuring unit 200 may include a belt type configured to surround the head of the subject, the EEG-measuring unit 200 may further include a first fixing portion 217a and a second fixing portion 217b for stably fixing the main frame 210 to the head of the subject. The first fixing portion 217a may be configured to connect a left portion and a right portion of the wearing unit 215 at both sides of the head of the subject with each other. The second fixing portion 217b may be configured to connect the first fixing portion 217a with the main frame 210. For example, the second fixing portion 217b may be configured to cross over a crown and a parietal lobe of the head. Therefore, the first fixing portion 217a and the second fixing portion 217b may be intersected with each other.

The EEG-detecting electrodes 220a-220d may be electrically connected with the controller 260. The EEG-detecting electrodes 220a-220d may simultaneously detect EEGs of at least one position, for example, four positions on the head of the subject. The EEG-detecting electrodes 220a-220d may be attached to the head of the subject by a non-invasive manner. For example, the EEG-detecting electrodes 220a-220d may include a dish type electrode or a snap type electrode.

As shown in FIG. 4, the EEG-detecting electrodes 220a-220d may measure the EEGs of a frontal lobe and the occipital lobe related to a neural network of a cerebrum cortex being in charge of recognition, perception and learning in the brain. A plurality of each of the EEG-detecting electrodes 220a-220d may be attached to the frontal lobe and the occipital lobe to measure the EEGs of each of the frontal lobe and the occipital lobe. In example embodiments, the two EEG-detecting electrodes 220a-220d may be provided to each of the frontal lobe and the occipital lobe.

The first and second EEG-detecting electrodes 220a and 220b may measure the EEG of the frontal lobe. The first and second EEG-detecting electrodes 220a and 220b may be located at a position of the face contact portion 214 in the main frame 210 corresponding to a forehead. Alternatively, the first EEG-detecting electrode 220a may be fixed to an additional fixing member connected to the main frame 210. The additional fixing member may be configured to make contact with the forehead of the subject.

The third and fourth EEG-detecting electrodes 220c and 220d may measure the EEG of the occipital lobe. The third and fourth EEG-detecting electrodes 220c and 220d may be installed at the first support 215a of the wearing unit 215. The third and fourth EEG-detecting electrodes 220c and 220d may make contact with the occipital lobe of the subject.

The reference electrode 230 may be electrically connected with the controller 260. The reference electrode 230 may be operated as a ground electrode. For example, the reference electrode 230 may be fixed to a skin of the subject. The reference electrode 230 may be electrically connected with the EEG-detecting electrodes 220a-220d. In example embodiments, as shown in FIG. 6, the reference electrodes 230 may be attached to an ear, particularly, an earlobe of the subject.

The EEG-measuring apparatus 200 may measure the EEG using a mono-polar derivation for amplifying a potential difference between each of the EEG-detecting electrodes 220a-220d and the reference electrode 230. In example embodiments, the EEG-detecting electrodes 220a-220d may be located at the positions corresponding to the frontal lobe and the occipital lobe. Alternatively, the EEG-detecting electrodes 220a-220d may include a plurality of electrodes configured to detect the EEGS of different portions on the head of the subject. Further, the EEG-detecting electrode may have additional channels, for example, eight channels.

The eye patch 240 may be inserted into the window of the main frame 210 to completely shut a sight of the eyes of the subject. The eye patch 240 may be supported by the main frame 210. The eye patch 240 may include an opaque material for shut the sight of the eyes.

When a stable EEG may be measured with the sight of the eyes being opened, the subject may generate an unconscious eye blinking by an external visual stimulus although the eyes of the subject may be closed. When the eye blinking may be generated, the brain may recognize the external visual stimulus because the sight of the eyes may be opened so that unintended artifacts may be reflected in the stable EEG.

According to example embodiments, because the EEG-measuring apparatus 200 may measure the stable EEG under the condition that the eye patch 240 may shut the sight of the eyes, the external visual stimulus may have no influence on the EEG of the subject although the unintended eye blinking may be generated.

The eye patch 240 may be configured to cover the eyes of the subject. Alternatively, when the window may be surrounded by the face contact portion 214 having the resilient member 214a, the eye patch 240 may be spaced apart from the face of the subject. In this case, although the eye blinking may be generated, feeling of irritation caused by the contact between the eye patch 240 and the eyes may be reduced.

The EEG-stimulating unit 250 may be arranged adjacent to the eye patch 240. The EEG-stimulating unit 250 may be configured to induce a transformation or a stimulus of the EEG of the subject. The EEG-stimulating unit 250 may provide the eyes of the subject with the visual stimulus. The EEG-stimulating unit 250 may include a light emitting element having a variable flickering period, for example, a light emitting diode (LED). The EEG-stimulating unit 250 may be installed at the face contact portion 214 corresponding to an inside portion of the eye patch 240. Alternatively, the EEG-stimulating unit 250 may be arranged in the second fixing portion 217b adjacent to the eye patch 240. The EEG-stimulating unit 250 such as the LED may have a high luminous characteristic in darkness. Thus, the EEG-stimulating unit 240 adjacent to the eye patch 240 may provide the eyes of the subject with the sufficient visual stimulus. Further, when the eye patch 240 may be spaced apart from the eyes of the subject due to the resilient member 214a of the main frame 210, a closed space may be formed between the eyes of the subject and the EEG-measuring apparatus 200. Thus, the EEG-stimulating unit 240 may be arranged in the closed space. Further, the closed space may provide the EEG-stimulating unit 240 with a sufficient luminous space so that the EEG stimulating may be effectively performed.

Further, the EEG-measuring apparatus 200 having the EEG-stimulating unit 250 may additionally perform a photonic driving response observation in which EEG changes caused by the visual stimulus may be measured as well as the stable EEG measured in the closed eyes. The photonic driving response observation may be performed under various flickering frequencies of the EEG-stimulating unit 250. For example, the photonic driving response observation may be performed under the flickering frequencies of about 3 KHz to about 30 KHz. Further, the flickering frequencies may be periodically increased by a multiple. The photonic driving response observation may be performed under a background frequency of the subject.

In a general photonic driving response observation, after measuring a stable EEG, a white flash lamp apart from the subject may be repeatedly turned-on and turned-off. According to example embodiments, the EEG-stimulating unit 250 may be arranged adjacent to the eye patch 240 so that the photonic driving response observation may be performed simultaneously with the EEG measurement.

The EEG stimulation on/off unit 255 may be installed at the main frame 210. The EEG stimulation on/off unit 255 may include a switch or a button on the main frame 210. The EEG-stimulating unit 250 may be driven in accordance with operations of the EEG stimulation on/off unit 255.

As shown in FIG. 5, the controller 260 may include an apparatus-controlling unit 260a, an EEG stimulation-controlling unit 260b and an EEG signal-generating unit 260c.

The apparatus-controlling unit 260a may be electrically connected with a power supply 272 and a reset unit 274. The power supply 272 may supply a power to the EEG-measuring apparatus 200. The reset unit 274 may reset the operations of the EEG-measuring apparatus 200. The apparatus-controlling unit 260a may control driving of the EEG-detecting electrodes 220a-220d and the reference electrode 230 by signals from the power supply 272 and the reset unit 274. The power supply 272 and the reset unit 274 may include a button or a switch on the main frame 210.

The EEG stimulation-controlling unit 260b may control the operations of the EEG stimulation on/off unit 255 and/or the EEG-stimulating unit 250. The EEG stimulation-controlling unit 260b may control the flickering period of the EEG-stimulating unit 250 and the driving of the EEG stimulation on/off unit 255 in accordance with control commands from the dementia-diagnosing apparatus 300. The system 100 for diagnosing and preventing the dementia including the EEG-measuring apparatus 200 may include the EEG-stimulating unit 250, the EEG stimulation on/off unit 255 and the EEG stimulation-controlling unit 260b so that an EEG of an abnormal subject may be synchronized with the EEG of the normal person.

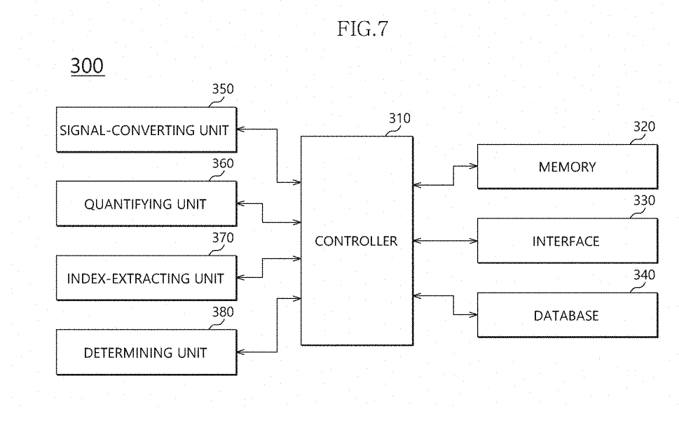

FIG. 7 is a block diagram illustrating an apparatus for diagnosing dementia in accordance with example embodiments.

Referring to FIG. 7, an apparatus 300 for diagnosing dementia may include a controller 310, a memory 320, an interface 330, a database 340, a signal-converting unit 350, a quantifying unit 360, an index-extracting unit 370 and a determining unit 380.

The controller 310 may be configured to control total operations of the dementia-diagnosing apparatus 300. For example, the controller 310 may include a central processing unit (CPU).

The memory 320 may be configured to store operational programs, application programs, control data, operational parameters, processing results, etc., of the dementia-diagnosing apparatus 300.

In example embodiments, the dementia-diagnosing apparatus 300 and the EEG-measuring apparatus 200 may be coupled with each other by a wire or wireless communication. The interface 330 may be communicated with the EEG-measuring apparatus 200.

Further, in order to allow a user for accessing to the dementia-diagnosing apparatus 300, the interface 330 may include an input interface including at least one of a keyboard, a mouse, a touch pad and a microphone, and an output interface including at least one of a display and a speaker.

The database 340 may be configured to store reference indexes, information of the subject, diagnosis results of the subject, etc. Here, the reference indexes may include stable EEG values of the normal person and a target frequency of a photo pattern. Particularly, the reference indexes may include a first reference index and a second reference index. The first reference index may include an alpha(α) wave peak level among peak levels by the EEGs of the normal person. The second reference index may include a theta (θ)wave peak level among the peak levels by the EEGs of the normal person. The reference index may include an EEG result of a photo stimulation to the normal person, i.e., the photonic driving response results as a reference senescence index.

The reference senescence index may correspond to quantified values of reactions of the background EEG, an alpha(α)-blocking, an existence of the photonic driving response, an increase of a gusted abnormal wave, induction frequency of the gusted abnormal wave, etc. For example, accumulated values of the gusted abnormal waves may be used as the reference senescence index. When the photonic driving response may be observed, the photonic driving response may be actively generated by the flash stimulation having a frequency of about 6 Hz to about 15 Hz approximate to the alpha(α) wave having the frequency of about 8 Hz to about 12 Hz dominant in the background activity. In contrast, when the photonic driving response in other frequencies may be observed, abnormal brain of the subject may be checked using the senescence index.

Further, the photonic driving response having an appearance frequency lower than an average frequency or reduced complexity of the photonic driving response may be checked as the senescence index.

The reference senescence index may be set by multiply checking the above-mentioned check items. Further, the reference senescence index may be set by concentratedly observing specific items among the check items.

The signal-converting unit 350 may be configured to convert the EEG signal of the subject as time series data provided from the EEG-measuring apparatus 200 into a signal of a frequency region. The signal-converting unit 350 may use a Fast Fourier Transform (FFT). However, the signal-converting unit 350 may use other techniques.

The EEG signal may be obtained from the front lobe, the occipital lobe, the temporal lobe and the parietal lobe of the subject in the stable state and the photo stimulation state. The signal-converting unit 350 may convert the EEG signals from each of the regions into the signal of the frequency region.

The EEG signals by the regions may be classified into the δ wave of no more than about 4 Hz, the theta(θ) wave of about 4 Hz to about 8 Hz, the alpha(α) wave of about 8 Hz to about 12 Hz, the beta(β) wave of about 12 Hz to about 30 Hz and the gamma(γ) wave of about 30 Hz to about 50 Hz.

Thus, the signal-converting unit 350 may convert the signal of the frequency region in accordance with the EEG signal in the stable state and the EEG signal in operating the EEG-stimulating unit 250 into the signal of the frequency region.

The quantifying unit 360 may be configured to extract an absolute power spectrum from the signal of the frequency region with respect to the regions of the brain converted by the signal-converting unit 350. In example embodiments, the quantifying unit 360 may integrate heights of a graph in each of the frequency regions with respect to the signals of the frequency regions from the regions of the brain transformed by the FFT to extract the absolute power spectrum. Therefore, the absolute power spectrum may reflect amplitudes and bandwidths of each of the frequencies.

Further, the quantifying unit 360 may quantify results of the photonic driving response observations by the EEG-stimulating unit 250 such as reactivity of the background EEG, the α-blocking, the existence of the photonic driving response, the increase of the gusted abnormal wave, the induction frequency of the gusted abnormal wave, etc.

The index-extracting unit 370 may be configured to extract a first index based on the alpha(α) waves, which may be greatly different between the abnormal subject and the normal person, from the absolute power spectrum extracted by the quantifying unit 360. Further, The index-extracting unit 370 may be configured to extract a second index based on the theta(θ) waves of the abnormal subject, which may be higher than that of the normal person, from the absolute power spectrum extracted by the quantifying unit 360. The first index and the second index may be represented by a following formula 1.

First index=standard alpha(α) wave peak level−OFFSET1

Second index=(standard alpha(α) wave absolute power value/standard theta(θ) wave absolute power value)−OFFSET2 [formula 1]

The index-extracting unit 370 may calculate the senescence indexes of the subject in accordance with the photonic driving response observation by the EEG-stimulating unit 250.

The determining unit 380 may compare the first index extracted from the index-extracting unit 370 with the first reference index, and the second index extracted from the index-extracting unit 370 with the second reference index to diagnose the dementia of the subject.

Further, the determining unit 380 may compare the brain senescence index in accordance with the photo driving observation results of the subject with the reference senescence index of the normal person to diagnose the dementia of the subject.

Method of Measuring EEG

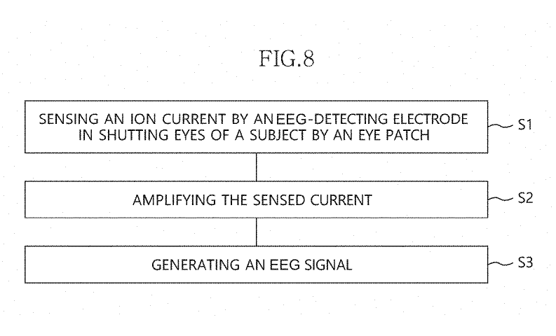

FIG. 8 is a flow chart illustrating operations for an EEG-measuring apparatus in accordance with example embodiments.

Referring to FIG. 8, the eyes of the subject may be shut using the eye patch 240. The power supply 272 may be operated to perform the measurement of the EEG.

In step S1, the EEG-detecting electrodes 220a-220d may sense the ion current from the brain of the subject.

In step S2, the EEG signal-generating unit 260c of the controller 260 may amplify the potential difference between the EEG-detecting electrodes 220a-220d.

In step S3, noises in the amplified potential difference may be filtered to generate the EEG signals.

Particularly, the EEG-detecting electrodes 220a-220d may sense the microcurrent from the brain of the subject to output the output signals having very low impedance. The output signals from the EEG-detecting electrodes 220a-220d may be applied to a differential amplifier in the EEG signal-generating unit 260c without unbalance of the impedance to generate the EEG signals.

Method of Diagnosing Dementia

FIG. 9 is a flow chart illustrating operations for a dementia-diagnosing apparatus in accordance with example embodiments.

Referring to FIG. 9, in step S11, the reference index may be set.

The reference index may include the first reference index and the second reference index. The first reference index may be obtained by applying a first offset, for example, about −15% to the α wave peak level. The second reference index may be obtained by applying a second offset, for example, about −20% to a ratio between the absolute power value of the 0 wave and the absolute power value of the α wave. Further, the reference index may include the reference senescence index based on the photonic driving response results when the photo stimulation may be applied to the normal person.

In step S12, the EEG-measuring apparatus 200 with the eye patch may receive the EEG signals to analyze the absolute power spectrum of the EEG signals. The signal-converting unit 350 of the dementia-diagnosing apparatus 300 may convert the EEG signals using the FFT into the signals of the frequency region. The signal-converting unit 350 may integrate the signals of the frequency regions by the waves to obtain the absolute power spectrum. The index-extracting unit 370 may extract the first and second indexes of the subject from the absolute power spectrum.

In step S13, after analyzing the EEG signals, the first index as the alpha(α) wave peak level of the subject may be checked whether the first index may be within the first reference index or not.

When the first index may be within the first reference index, in step S14, the second index as the ratio between the absolute power value of the α wave and the absolute power value of the theta(θ) wave may be checked whether the second index may be within the second reference index or not.

When the second index may be within the second reference index, in step S15, the brain of the subject may be diagnosed to be normal.

When the first index may not be within the first reference index, in step S16, the subject may be diagnosed as a patient with the dementia.

When the second index may not be within the second reference index, in step S17, although the subject may not be the patient with the dementia, a dementia caution may be notified to the subject.

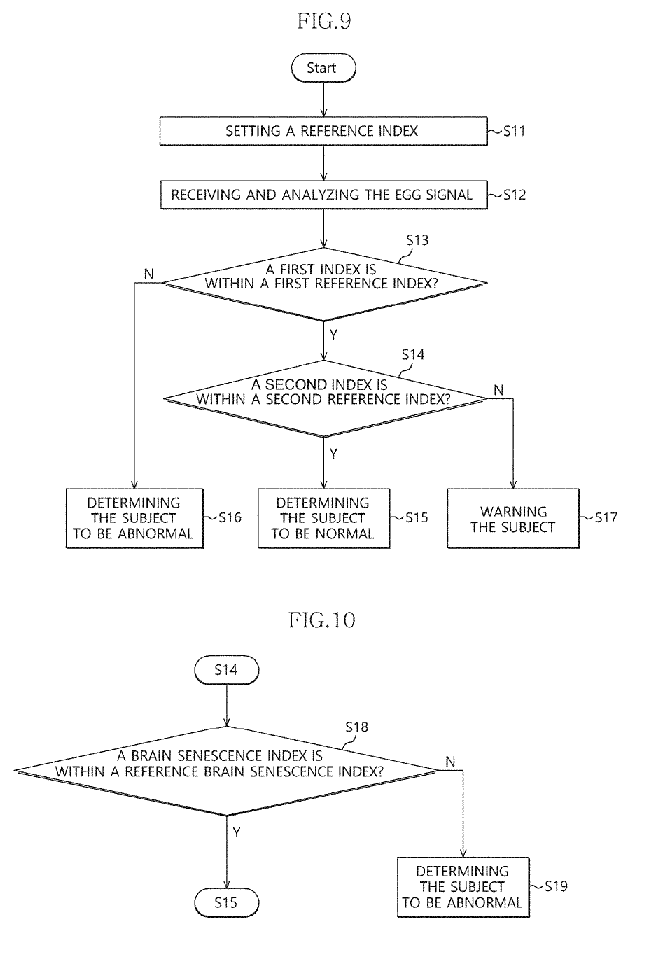

FIG. 10 is a flow chart illustrating operations for a dementia-diagnosing apparatus in accordance with example embodiments.

Referring to FIG. 10, the photonic driving response results may be additionally checked whether the photonic driving response results may be within the reference brain senescence index or not between the step S14 and the step S15.

When the photonic driving response results may be within the reference brain senescence index, in step S15, the subject may be determined to be normal. When the photonic driving response results may not be within the reference brain senescence index, in step S19, the subject may be determined to be abnormal. That is, the subject may be determined as the brain senescence group. The brain senescence group may be classified into the abnormal group together with the latent dementia group and the cautious dementia group. The dementia caution may be notified to the brain senescence group.

The method of diagnosing the dementia may be performed using an application in a user's terminal such as a personal computer, a smart phone, a tablet PC, a notebook, etc.

Method of Preventing Dementia

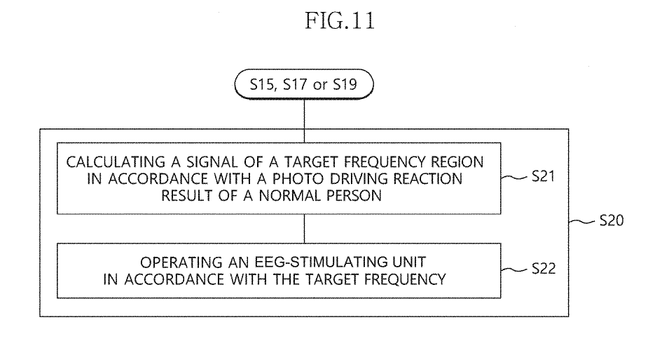

FIG. 11 is a flow chart illustrating operations for a dementia-diagnosing and preventing system in accordance with example embodiments.

Referring to FIGS. 9 to 11, in step S20, preventive operations may be performed to the abnormal subjects included in the steps S15, S17 and S19 using the photo stimulation.

The step S20 may include step 21 in which the signal of the target frequency region in accordance with the photonic driving response results may be set, and step S22 in which the EEG-stimulating unit 250 may be driven in response to the signal of the target frequency region with respect to the abnormal subject.

The signal-converting unit 350 may set the signal of the target frequency region. The signal of the target frequency region generated from the signal-converting unit 350 may be transmitted to the EEG stimulation-controlling unit 260b of the EEG-measuring apparatus 200 in accordance with the determined results of the determining unit 380 as the control command or the control signal.

The EEG stimulation-controlling unit 260b may control the drive and the flickering period of the EEG stimulation on/off unit 255 and the EEG-stimulating unit 250 to correspond the EEG-stimulating unit 250 to the signal of the target frequency region. Thus, the abnormal subject may receive the photo stimulation or the visual stimulation corresponding to the target frequency from the EEG-stimulating unit 250.

When the photo stimulation may be applied to the EEG, the EEG may generate a temporal EEG coherence to the photo stimulation. Therefore, when the photo stimulation corresponding to the photonic driving response results of the normal person may be applied to the abnormal subject, the EEG of the abnormal subject may be synchronized with the EEG of the normal person so that the EEG of the abnormal subject may be temporarily matched with the EEG of the normal person. When the above-mentioned stimulation may be repeated, the EEG of the abnormal subject may be closely synchronized with the EEG of the normal person to prevent and cure the dementia.

According to example embodiments, the stable EEG may be measured under the condition that the eyes of the subject may be shut by the eye patch. Thus, although the eye blinking may be generated, the visual image from the outside may be shut so that the artifacts may not be mixed in the EEG.

Further, the EEG-stimulating unit may be positioned adjacent to the eye patch. Therefore, the photo stimulation in accordance with the photonic driving response results of the normal person may be applied to the abnormal subject so that the EEG of the abnormal subject may be synchronized with the EEG of the normal person to prevent the dementia.

The above embodiments of the present disclosure are illustrative and not limitative. Various alternatives and equivalents are possible. The examples of the embodiments are not limited by the embodiments described herein. Nor is the present disclosure limited to any specific type of semiconductor device. Other additions, subtractions, or modifications are obvious in view of the present disclosure and are intended to fall within the scope of the appended claims.

What is claimed is:

1. An apparatus for measuring an electroencephalogram (EEG), the apparatus comprising:
at least one EEG-detecting electrode attached to a head of a subject to measure the EEG of the subject; and
an eye patch configured to shut an eye view of the subject in measuring the EEG.

2. The apparatus of claim 1, further comprising a main frame configured to support the eye patch and supported by a face of the subject.

3. The apparatus of claim 2, wherein a portion of the main frame configured to make contact with the face of the subject comprises a resilient member detachably attached to the main frame.

4. The apparatus of claim 2, further comprising a wearing unit connected with the main frame and configured to fix the main frame to the face of the subject.

5. The apparatus of claim 4, wherein the wearing unit is configured to partially surround an occipital lobe of the subject.

6. The apparatus of claim 5, further comprising:
a first fixing portion configured to connect a portion of the wearing unit left the head of the subject with a portion of the wearing unit right the head of the subject; and
a second fixing portion configured to connect the main frame with the first fixing portion and extended over a parietal lobe of the head.

7. The apparatus of claim 6, wherein the EEG-detecting electrode comprises:
first and second EEG-detecting electrodes configured to measure EEGs of a frontal lobe of the subject; and
third and fourth EEG-detecting electrodes configured to measure EEGs of the occipital lobe of the subject.

8. The apparatus of claim 7, wherein the first and second EEG-detecting electrodes are arranged in the main frame corresponding to the frontal lobe of the subject, and the third and fourth EEG-detecting electrodes correspond to the occipital lobe of the subject.

9. The apparatus of claim 1, further comprising a reference electrode electrically connected with the EEG-detecting electrode, the reference electrode configured to make contact with a skin of the subject to be earthed.

10. The apparatus of claim 1, further comprising an EEG-stimulating unit configured to induce a transformation of the EEG of the subject.

11. The apparatus of claim 10, wherein the EEG-stimulating unit comprises a light emitting element arranged adjacent to the eye patch to provide the eyes of the subject with a visual stimulus.

12. The apparatus of claim 11, further comprising a controller configured to sense the EEG of the subject from the EEG-detecting electrode to generate an EEG signal and control a flickering period of the EEG-stimulating unit in response to a photonic driving response results of a normal person.
